(19) United States
(12) Patent Application Publication
(10) Pub. No.: US 2025/0064435 A1
(43) Pub. Date: Feb. 27, 2025

(54) Method For Cell Treatment

(71) Applicant: Jes Tougaard Gram, Scottsdale, AZ (US)

(72) Inventor: Jes Tougaard Gram, Scottsdale, AZ (US)

(21) Appl. No.: 18/454,710

(22) Filed: Aug. 23, 2023

(51) Int. Cl. A61B 10/02; A61B 90/00

(52) U.S. Cl. CPC A61B 10/0275; A61B 90/37; A61B 2090/378

(57) ABSTRACT

The present invention concerns a method of extended contactless treatment of targeted cells using at least one inductive heat-able and/or a slow dissolving time release drug delivery part. Employing inductive contactless heating of least one inductive heat-able material/compounded part and/or a slow dissolving time release drug delivery part introduced into the body of a living being, can enable a contactless extended targeted cell treatment after the delivery procedure. In combinations with other treatments where induced current by an inductive heating device and/or a slow dissolving time release drug delivery part can help remove/weaken target cells e.g. by means of at least one inductive heat able part capable of a controlled heating by a contactless induction heating device for hyperthermia treatment of cancer and/or a slow releasing targeted chemo treatment delivered directly in a cancer tumor.

### FIELD OF INVENTION

The present invention relates to a method for interacting with targeted cells and/or targeted drug delivery. More particularly, the present invention relates to a method for introducing particles into the body preferably through a biopsy procedure.

### BACKGROUND OF THE INVENTION

Patients suspected of having a disease with potentially harmful treatments, such as cancer, a small sample of tissue called biopsy, is needed to make a correct diagnosis. In most cases, a biopsy is used to identify specific cancer characteristics to allow for a more personalized therapy. Samples of tissue often need to be harvested in order to perform a diagnostic and specialized testing.

Currently, standard core needle biopsies require multiple passes through tissue to collect enough tissue cores. However, more passes by the needle increases the likelihood of complications such as over bleeding or even death.

In addition, an increased number of passes may mean longer procedural times and longer post-procedural monitoring. Another means of obtaining more tissue is the use of a larger needle, but bigger needles also carry increased risk of complications, such as bleeding or other organ-specific complication.

An US patent application 20090143808 on Guided tissue cutting device, method of use and kits therefore assigned to Russell A. Houser. The prior art describes a guided tissue cutter configured to receive a cutter adapted to cut a target. The cutter is adapted to cut a target tissue and heat may be applied via insertion of a heated probe. But the application still lacks the ability of delivering macroparticles in the cavity made by the biopsy device.

An U.S. Pat. No. 11,607,205 on Biopsy system for enhanced tissue harvesting assigned to Michael C. Larson. The prior art describes a biopsy system for harvesting larger volumes of tissue. Inserting the biopsy needle inside patient for cutting the tissue. The invention discloses about retracting the sheath to expose the cutting mechanism. The needle includes an upper layer-sheath & inner layer and cut the tissue sample from any electrical or thermal damage. The prior art still fails to describe the use of inductive heating and delivery of macroparticles or larger particles.

Another JP patent 4598770 on Biopsy needle system assigned to Barry N. Gellman. The prior art describes a needle biopsy system for marking a sampling site. The biopsy needle system includes a stylet and cannula. The stylet and cannula are relatively movable so that the stylet is positioned above the sampling area. Moreover, the sampled area can be treated by tissue ablation treatment using induction heating. Still, the prior art discloses the delivery of a marker and lacks the use of macroparticles for extended contactless treatment of targeted cells.

The Prior approaches used nanoparticles to transfer heat to cancer cells. Since nanoparticles cannot usually be uniformly sized, it is difficult to accurately regulate temperature and location of treatment.

Therefore, there is a need for developing a method or a system for targeted cell treatment where the inductive heat is distributed uniformly and/or a time release drug compound. So, the use of macroparticles preferably delivered during a biopsy procedure in the area the targeted cells is suggested.

Although, image-guided biopsy combines an imaging procedure such as a CT scan, MRI or ultrasound with a biopsy needle and can be used as delivery device of the heat-able and/or time release drug delivery part. An alternative to the biopsy needle could be an endoscope procedure. The endoscope can be inserted through your mouth, rectum, urinary tract or a small incision in your skin, and the endoscope having a flexible tube with a camera to see structures inside the body capable of introducing special tools through the tube to deliver the heat-able and/or time release drug delivery part at the desired destination. Another alternative could be a surgical incision to access the suspicious area of cells to deliver the heat-able and/or time release drug delivery part at the desired destination.

The use of macroparticles provide uniform time release of a drug and/or distribution of the inductive heat and providing accuracy in the process.

It is apparent now that numerous methods and systems are developed in the prior art that are adequate for various purposes. Furthermore, even though these inventions may be suitable for the specific purposes to which they address, accordingly, they would not be suitable for the purposes of the present invention as heretofore described. Thus, a method or a system for targeted cell treatment where the inductive heat is distributed uniformly through the use of macroparticles.

Induction heating is the process of heating electrically conductive materials, namely metals or semi-conductors, by electromagnetic induction, through heat transfer passing through an induction coil that creates an electromagnetic field within the coil and/or proximity to the coil to heat up steel and stainless steel, brass, graphite, tungsten, gold, silver, aluminum, carbide, copper and copper alloys, chrome, nickel, and nickel alloys.

An induction heater consists of an electromagnet and an electronic oscillator that passes a high frequency alternating current (AC) through the electromagnet. The rapidly alternating magnetic field penetrates the object, generating electric current inside the conductor called eddy currents. The eddy currents flow through the resistance of the material and heat it by Joule heating. In ferromagnetic and ferrimagnetic materials, such as iron, heat also is generated by magnetic hysteresis losses.

Compounded materials having a metal like iron, copper, aluminum, steel, or brass could also be made of a semiconductor such as graphite, carbon, silicon carbide, metal oxide or a compounded material that also contain nonconductive components.

The frequency of the electric current used for induction heating depends on the object size, material type, coupling (between the work coil and the object to be heated), and the penetration depth. An important feature of the induction heating process is that the heat is generated inside the object itself, instead of by an external heat source via heat conduction.

In medicine, a stent is any device which is inserted into a blood vessel or other internal duct to expand it to prevent or alleviate a blockage. Traditionally, such devices are fabricated from metal mesh and remain in the body permanently or until removed through further surgical intervention. A bioresorbable stent (also called bioresorbable scaffold, biodegradable stent or naturally dissolving stent) serves the same purpose but is manufactured from a material that may dissolve or be absorbed in the body.

Bioabsorbable metals are based on magnesium (Mg), iron (Fe) and zinc (Zn), as their degradation products are biocompatible, magnesium alloys being the most popular. They have been used to develop bioabsorbable structural implants, including plates, screws, and bone anchors. Ceramics are non-metallic, often crystalline oxides, of commonly nitride or carbide materials. Bioabsorbable ceramics include a variety of materials, such as calcium and carbon phosphates, alumina, and hydroxyapatite (HAp). Ceramics could be used successfully for coatings on and/or in conductive blends made implants to aid fixation of protein coatings. These may come in the form of porous or nonporous blocks, granules, injectable compositions, and custom designs.

Temperature interferes with the tumor's ability to repair itself e.g., on the effects of radiation and enhances the effectiveness of chemotherapy drugs. Heat increases blood flow to the tumor, making cancer cells more sensitive to radiation and chemotherapy. Heat enhances the body's immune response against cancer cells.

Thus, knowing what temperature cancer cells die at, will be the basis for adjusting the appropriate temperature in the treatment of cancer patients. Instead of completely killing cancer cells, the impact of moderate heat will make these malignant cells weaker, so they are easier to destroy by radiation and chemotherapy. High temperatures also make mutant cells that are resistant to radiation and chemotherapy more susceptible.

Malignant particles have a special sensitivity to high temperatures, that's why we develop fever when we have infections, but if the temperature is too high, it will affect normal benign cells. To apply in cancer treatment, doctors will use local heating with a temperature between 42° C. and 43° C., which is the optimal temperature to inhibit and complement other therapies. But for temperatures above 43° C., not only the cancer cells die, but the patient's life is also threatened due to protein denaturation caused by excessive heat. In addition, excessive temperature increase can cause shock and local trauma to the patient.

It has been long recognized that hyperthermia in the 40-47° C. temperature range kills cells in a reproducible time and temperature dependent manner. Survival curves for temperatures in the 43-47° C. range typically show a shoulder with an exponential reduction in clonogenicity survival as a function of time at a given temperature.

In contrast, cell survival curves for temperatures of 42.5° C. and below, depending on the cell line, will show a shoulder, an exponential portion of cell killing followed by a plateau in cell killing, due to the development of chronic thermal tolerance. The principal conclusion from these studies is that for hyperthermia, thermal dose is a combination of time and temperature.

Different types of cancer develop in different ways, but all of them begin with uncontrolled cell division. To produce a new cell, an existing cell must duplicate its genetic material (DNA) and then divide. Cancer develops when the cell's genetic material becomes damaged, or ‘mutated’. The mutations cause the cells to divide uncontrollably. Cancer cells gradually accumulate within the organ in which the growth began, eventually forming a tumor.

In a new study, researchers at the Centre for Cancer Biomedicine have discovered how cancer cells grow. Using fruit flies, they found that cancer cells grow by extracting nutrients from their surroundings. These findings may have an impact on bow cancer is treated.

Cancer cells have an increased need for nutrients and therefore adopt different strategies to access macromolecules from the tumor microenvironment. Nutrient scavenging consists in the uptake of macromolecules from the extracellular environment and their degradation to produce the cellular energy molecule, adenosine triphosphate (ATP) or to be used in anabolism.

Cancer cells require extra energy throughout the growth process, and so change their energy consumption compared with healthy cells. When their healthy neighbors break down proteins into amino acid building blocks, the cancer cells absorb these and use them to grow.

The cancer cells thus trick their neighbors into supplying them with energy and make use of sugars and amino acids from the bloodstream to grow and divide indefinitely. The results of the study are similar to those obtained in studies of human cells. Researchers at Harvard have demonstrated the transfer of amino acids between healthy neighboring cells and cancer cells, in tissues from patients with pancreatic cancer.

A Harvard study found that neighboring cells break down their own proteins through the process of autophagy or ‘self-eating’, old and damaged cells are broken down, removed and release amino acids, which the cancer cells then absorb and use. However, the cancer cells were unable to grow if the researcher's blocked autophagy in neighboring cells or prevented the cancer cells from absorbing the amino acids. This important finding may lead to the development of new cancer treatments.

During cancer progression, tumor cells go through different stages, which are defined as hallmarks of cancer. One of the main hallmarks is the ability to sustain proliferation. Mis-regulation of growth-promoting signals stimulates cell survival and energy metabolism, resulting in tumor growth.

As cancer further develops, cells may become able to disseminate from the primary site of origin. Genes that in normal tissues express molecules involved in cell-to-cell adhesion and cell-to-extracellular matrix adhesions are altered in highly aggressive carcinomas, typically downregulated. After losing cell-cell adhesions, cancer cells acquire migratory ability, leading eventually to invasion into neighboring tissues and forming metastatic sites in distant organs.

During these transitions, cancer cells undergo metabolic changes, which allow them to satisfy their increased need of energy. The reprogramming of energy metabolism is now recognized as one of the hallmarks of cancer. One of the most known metabolic adaptations in malignant cells is the Warburg effect, that is characterized by increased glucose uptake and lactate production in the presence of oxygen.

Dormant cancer cells are one of the most threatening aspects of cancer and can lead to reoccurrence of metastatic tumors after a long period of latency. Tumor cell dormancy can be induced by nutrient deprivation but the mechanism behind the revival of the dormant cells remains mainly elusive.

Up till now most time release drug administration has been in oral form with the huge challenge of passing through the digestive system that aggressively breaks down large parts of most drug administration and also have a limited operation window before it leaves the body again.

Time-release drugs use a special technology to release small amounts of the medication into a person's system over a long period of time. This is also referred to as sustained release, extended release, or controlled release. These tend to come in pill form and are simply made to be more potent but dissolve slowly. Sustained release technology is a class of technology characterized by slowly releasing specific active substances into a target medium to keep a certain concentration in the system within valid time.

Most time-release drugs are now formulated with the active pharmaceutical ingredient embedded in a matrix of insoluble materials such as acrylics or chitin. Here the mechanism relies upon the dissolving drug finding its way out through pores. Some sustained release drug forms dissolve the active into a matrix.

The basic mechanisms that control the release of the drug molecules through the polymeric layer are osmosis, diffusion, chemical degradation, swelling and dissolution, with diffusion playing a dominant role in many controlled release systems.

A number of controlled drug release approaches deliver drug at a steady rate into an extravascular region of the body. Examples of this type of drug delivery include transdermal patches, implantable pumps, and intramuscular depot injections.

The drug delivery system enables the release of the active pharmaceutical ingredient to achieve a desired therapeutic response. Conventional drug delivery systems (tablets, capsules, syrups, ointments, etc.) suffer from poor bioavailability and fluctuations in plasma drug level and are unable to achieve sustained release.

Without an efficient delivery mechanism, the whole therapeutic process can be rendered useless. Moreover, the drug has to be delivered at a specified controlled rate and at the target site as precisely as possible to achieve maximum efficacy and safety. Controlled drug delivery systems are developed to combat the problems associated with conventional drug delivery.

Drug delivery has the potential to have a tremendous impact on treatment of retinal diseases. There are a large number of drugs that are reasonably effective to treat retinal conditions, but those drugs are limited by delivery issues such as the need to have the molecule cross the blood-eye barrier, be present for long times, or the need to mitigate side-effects.

The challenges of having drugs at a physiologically relevant concentration for extended periods or in a localized delivery system are challenges that can be solved with drug delivery technology, whether it is using cellular delivery systems, microelectromechanical based devices, polymer matrices, or gene delivery systems.

Drug delivery technologies have been proven to improve treatment outcomes in many ways, including enhancing therapeutic efficacy, reducing toxicity, increasing patient compliance, and enabling entirely new medical treatments. As the therapeutic landscape has evolved from small-molecule drugs to a new generation of therapeutics including proteins, peptides, monoclonal antibodies, nucleic acids, and even live cells, drug delivery technologies have also evolved to meet their unique delivery needs.

Radiation therapy can treat many different types of cancer. It can also be used in combination with other cancer treatments, such as chemotherapy and/or surgery. Cancer begins when healthy cells change and grow out of control. All cells in the body go through a cycle to grow, divide, and multiply. Cancer cells go through this process faster than normal cells. Radiation therapy damages cell DNA so the cells stop growing or are destroyed.

Unlike other cancer treatments affect the whole body, such as chemotherapy, radiation therapy is usually a local treatment. This means it generally affects only the part of the body where the cancer is located. Some healthy tissue near the cancer cells may be damaged during the treatment, but it usually heals after treatment ends. There are many different types of radiation therapy, and they all work a little bit differently to destroy cancer cells.

The goals of radiation therapy depend on your type of cancer and if and how far it has spread. Radiation therapy can be given alone or as a part of a treatment plan that includes different treatments. Often, the goal of radiation therapy is to get rid of all the cancer and keep it from coming back. Radiation therapy can be given before other treatments, such as surgery, to shrink a large tumor. This is called “neoadjuvant radiation therapy.”

Radiation therapy can be given after other kinds of treatments to destroy any remaining cancer cells. This is called “adjuvant radiation therapy.” Radiation therapy can be used to relieve the signs and symptoms of cancer. This is called “palliative radiation therapy.”

Radiation therapy can be used to treat many different types of cancer. More than half of people with cancer will receive some type of radiation therapy. For some cancers, radiation therapy alone is an effective treatment. Other types of cancer respond best to a combination of treatments. Radiation therapy can also be used to treat recurrent cancer and metastatic cancer.

Internal radiation therapy is also called brachytherapy. This type of radiation therapy is when radioactive material is placed into the cancer or surrounding tissue. Implants may be permanent or temporary. This treatment may require a hospital stay.

The different types of internal radiation therapy include permanent implants. These are tiny steel seeds that contain radioactive material. The capsules are about the size of a grain of rice. They deliver most of the radiation therapy around the implant area. However, some radiation may exit the patient's body. This requires safety measures to protect others from radiation exposure. Over time, the implants lose radioactivity. The inactive seeds remain the body.

Brachytherapy is a type of internal radiotherapy. A small radioactive material called a source is put into your body, inside or close to the cancer. Or into the area where the cancer used to be before having surgery. There are different types of radioactive sources (also called implants) such as seeds, wires or discs. They deliver radiotherapy to the area, destroying the cancer cells. Healthy tissue near to the cancer gets a lot less radiation.

A doctor specialized in this kind of treatment works out how much radiation a patient need. This affects how long the radioactive source stays inside you for. This can be anything from several minutes to a few days or can stay in place permanently. If the source stays inside, you permanently, it stops giving off radiation after a few weeks or months.

### SUMMARY OF THE INVENTION

The present invention concerns a method of extended contactless treatment of targeted cells using at least one inductive heat-able and/or a slow dissolving time release drug delivery part. A method that employs inductive contactless heating of least one inductive heat-able material introduced into the body of a living being enabling contactless extended targeted cell treatment after the delivery procedure.

Or in combinations with other treatments where induced current by an inductive heating device can help remove/weaken target cells by means of at least one inductive heat able part capable of a controlled heating by a contactless induction heating device, e.g., for hyperthermia treatment of cancer.

Such an inductive heat-able part could be any size and/or formed parts, including any solid, hollow and/or compounded materials including bioabsorbable, bio-reabsorbable metals and metal alloys used e.g. in temporary biomedical implants, but also as a part of a time release drug delivery carrier e.g. a mixed compound.

Induction heating is an accurate, fast, repeatable, efficient, non-contact technique for heating metals or any other electrically conductive materials.

In a preferred embodiment an inductive heat-able part is introduced into the cavity where a biopsy sample has been removed from a tumor.

Using a biopsy procedure to make one or more cavities in a tumor would enable personalized inductive heated hyperthermia treatment of any cell growth or infection in the body without delay without stressing the rest of the patient's body.

The conductive part could also be coated in a specific protein or other kind of bait e.g., amino acid and/or sugars targeted e.g., at cancer cells and applied directly into e.g., as a stent into a blood vessel for leukemia and/or directly into a cancer tumor where the cancer cells that then would try to engulf the bait and be ready for cell interaction when exposed to induction heat.

This method and device could also be used as a medical treatment delivery vehicle system to deliver a time release drug compound such as targeted chemotherapy using a slow dissolving time release drug delivery part or a localized radiation treatment part, potentially combined with the inductive heated hyperthermia treatment.

The use of uniformly sized macro particles on as the heat-able elements (such as the size of a grain of rice), rather than nanoparticles allows the more accurate regulation of temperature and position of treatment. Preferably the hyperthermia treatment is conducted contactless at least once at a temperature between 42° C. and 43° C., which is the optimal temperature to inhibit and complement other therapies.

Care must be taken for temperature treatments above 43° C., since not only the cancer cells die, but the patient's life is also threatened due to protein denaturation caused by excessive heat. In addition, excessive temperature increase can cause shock and local trauma to the patient.

The inductive heat able particles preferable sized to be detectable by Ultrasound, MRI and/or CT scanner and a thermal imager/sensitive device for location and distance and e.g. an infrared camera and thereby also contactless temperature detectable and thereby controllable after being introduced into the body.

Having self-controlling low Curie temperature magnetic heat able part material for induction heating based on compounding mix. The magnetization decreases with increasing amount of additives thereby controlling the saturation magnetization. The compositions of e.g. alloys can be mixed to realize a suitable Curie temperature. Controlling size and material composition of the heat-able element along with distance to the inductive heat source and the level/strength of eddy current are important features.

Having a controllable temperature in the inductive heat-able part can also be used for heat-controlled release when combined with a drug delivery composition where a heighten temperature will make the drug delivery part sweat and/or release a drug dose.

The primary objective of the present invention is method for continues treatment of targeted cells over an extended period after delivering an extended treatment capable part in one or more cavities in e.g. a tumor with a surgical tool.

The method includes firstly the surgical tool is inserted within patient body preferable for collecting a biopsy sample before delivering an extended treatment capable part in the cavity.

Once the position of the surgical tool is confirmed through e.g. an ultrasound or other detection technology. Finally, the surgical tool is removed with the sample from the patient body and now the actual targeted hyperthermia cell treatment can be started by applying inductive heat via the heat-able part in at least one treatment session.

A noninvasive version of the invention comes in the form of a oral time release pill that is activated and/or opened by inductive heating, having a controllable temperature in a inductive heat-able part of the pill can also be used for heat-controlled release when combined with a drug delivery composition where a heighten temperature will make the drug delivery part sweat and/or release a drug dose. This could be done by raising the temperature in the time release drug delivery device releasing the drug dose by raising the pressure in the pill opening it by heat or melting a plug or parting line of the time release pill to release a drug dose. This version of the invention could also give the option not to release the drug dose, thereby only receiving a drug dose when needed. Targeted treatment could then be delivered at any specific point after the pill enters the body and until it leaves the body. The pill could pass through the hostile environment of the stomach to be released in the intestine at a specific preset time and/or by the pill position in the intestine for targeted treatment. Here the use of tracking devise like an Ultrasound, MRI and/or CT scanner combined with an inductive heat applicator would insure a targeted contactless treatment without any invasive procedure using surgical instruments.

Drug delivery and targeted treatments with antibiotic or pain reliver could be orally administered much more accurately if the pill is allowed to pass through the hostile environment of the stomach to be released in the intestine at a preset time and/or place.

Another objective of the present invention is a surgical tool for conducting a biopsy while delivering an extended treatment capable part in the cavity. The surgical tool comprises a needle portion and a plunger portion the needle portion capable of retrieving a sample and delivering an extended treatment capable part.

The needle portion includes a compartment for receiving a sample and a compartment for delivering a part during procedure. Where said part could be designed/formed to compliment said delivery compartment. In a special version of the invention and preferably where no biopsy sample is collected, the needle portion can be exposed to inductive heat during the procedure to help seal the incision wound and/or as an emergency option if e.g. if a blood vessel is ruptured during the procedure. Having this induction heating option of the biopsy needle might allow for a lager needle diameter and thereby lager sample and delivery options.

The plunger portion is connected to the base of the needle portion to control the movement of the needle portion inside the patient during the biopsy and part delivery procedure.

Other objectives and aspects of the invention will become apparent from the following detailed description, taken in conjunction with the accompanying drawings, which illustrate, by way for example, the features in accordance with embodiments of the invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

Although, the invention is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

The presence of broadening words and phrases such as “one or more,” “at least,” “but not limited to” or other like phrases in some instances shall not be read to mean that the narrower case is intended or required in instances where such broadening phrases may be absent.

### BRIEF DESCRIPTION OF DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa.

Furthermore, elements may not be drawn to scale. Non-limiting and non-exhaustive descriptions are described with reference to the following drawings. The components in the figures are not necessarily to scale, emphasis instead being placed upon illustrating principles. Furthermore, the drawings may contain text or captions that may explain certain embodiments of the present invention. This text is included for illustrative, non-limiting, explanatory purposes of certain embodiments detailed in the present invention. In the drawings:

Embodiments of the invention are described with reference to the following figures. The same numbers are used throughout the figures to reference like features and components. The features depicted in the figures are not necessarily shown to scale. Certain features of the embodiments may be shown exaggerated in scale or in somewhat schematic form, and some details of elements may not be shown in the interest of clarity and conciseness.

FIG. 1A illustrates a flowchart on method to conduct biopsy procedure in accordance with the present invention;

FIG. 1B illustrates a method for conducting biopsy procedure in accordance with the present invention;

FIG. 1C illustrates a method for conducting a biopsy procedure and insert delivery in accordance with the present invention;

FIG. 2A illustrates a surgical tool to conduct biopsy in accordance with the present invention;

FIG. 2B illustrates a side view of a needle portion of the surgical tool in accordance with the present invention; and

FIG. 2C illustrates a number of variations in delivery unit of the needle portion in accordance with the present invention.

### DETAILED DESCRIPTION OF THE INVENTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “units” represents the dimension in any units such as centimeters, meters, inches, foots, millimeters, micrometer and the like and forms thereof, are not necessarily limited to members in a list with which the words may be associated.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items from the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present invention contains headers. It should be understood that these headers are used as references and are not to be construed as limiting upon the subjected matter disclosed under the header.

This specification includes references to “one embodiment” or “an embodiment.” The appearances of the phrases “in one embodiment” or “in an embodiment” do not necessarily refer to the same embodiment. Particular features, structures, or characteristics may be combined in any suitable manner consistent with this disclosure.

The presence of broadening words and phrases such as “one or more,” “at least,” “but not limited to” or other like phrases in some instances shall not be read to mean that the narrower case is intended or required in instances where such broadening phrases may be absent.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

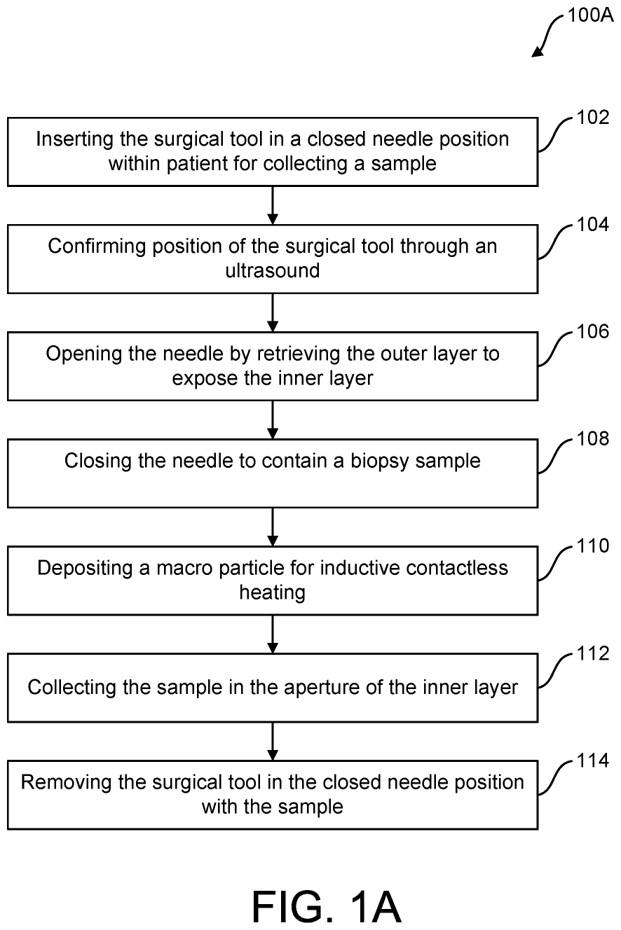

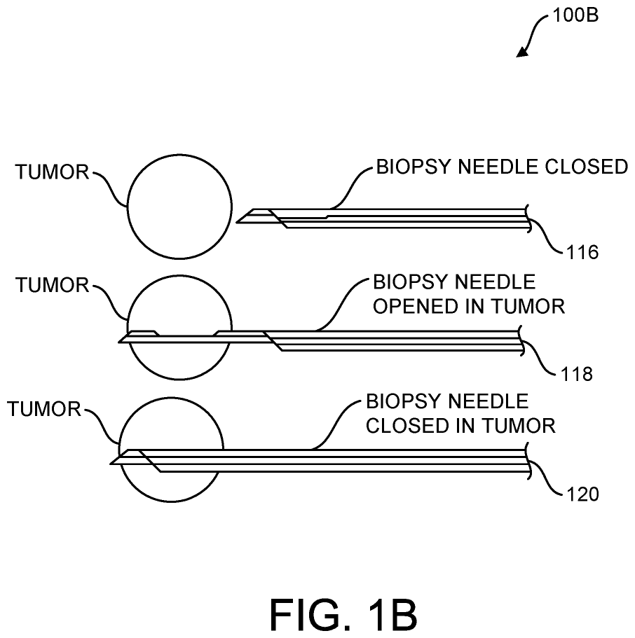

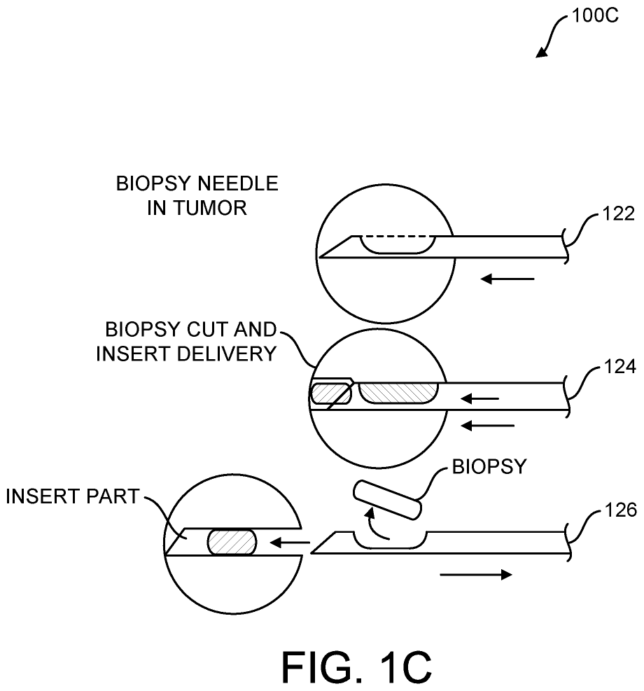

FIG. 1A illustrates a flowchart 100A on method to conduct biopsy procedure. A method to perform a biopsy to make one or more cavities in a tumor with a surgical tool. The method includes firstly the surgical tool is inserted within patient body for collecting a sample 102. The surgical tool includes an outer layer and an inner layer. Once, the position of the surgical tool is confirmed through an ultrasound 104. The needle is opened and the outer layer is retrieved to expose the inner layer 106.

Closing the outer layer to cut the biopsy sample from the tumor and deliver the part. The sample is collected in the aperture of the inner layer 112. Finally, the surgical tool is removed with the sample from the patient body 114.

A method of heat interacting with targeted cells. A method that employs inductive contactless heating 110 of least one inductive heat-able material introduced into the body of a living being as a standalone treatment. Or combinations with other treatments where induced current by an inductive heating device can help remove/weaken target cells by means of at least one inductive heat able part capable of a controlled heating by a contactless induction heating device, e.g., for hyperthermia treatment of cancer.

Such an inductive heat-able part could be any size and/or formed parts, including any solid, hollow and/or compounded materials including bio-reasorbable metals and metal alloys used e.g. in temporary biomedical implants.

Induction heating is an accurate, fast, repeatable, efficient, non-contact technique for heating metals or any other electrically conductive materials.

The conductive part could also be coated in a specific protein or other kind of bait e.g., amino acid and/or sugars targeted e.g., at cancer cells and applied directly into e.g., into the blood for leukemia and/or directly into a cancer tumor where the cancer cells that then would try to engulf them and be ready for cell interaction when exposed to induction heat.

This method and device could also be used as a medical treatment delivery vehicle system to deliver a time release drug/treatment part e.g. targeted chemotherapy or localized radiation treatment. This could be in combination with hyperthermia treatment.

The use of uniformly sized macro particles on as the heat-able elements (such as the signs size of a grain of rice), rather than nanoparticles allows the more accurate regulation of temperature and position of treatment.

The inductive heat able particles preferable sized to be detectable by Ultrasound, MRI and/or CT scanner and a thermal imager/sensitive device for location and distance and e.g. an infrared camera and thereby also contactless temperature detectable and thereby controllable after being introduced into the body.

Having self-controlling low Curie temperature magnetic heat able part material for induction heating based on compounding mix. The magnetization decreases with increasing amount of additives thereby controlling the saturation magnetization. The compositions of e.g. alloys can be mixed to realize a suitable Curie temperature.

FIG. 1B illustrates a method for conducting biopsy procedure 100B. The procedure includes inserting the surgical tool in a closed needle position 116. Once the needle tip reaches the target the needle is opened exposing the inner layer of the needle portion and a sample is removed from the target 118. The needle is again closed and then removed from the patient 120. While delivering the heat-able part in the cavity at the same time.

In a preferred embodiment an inductive heat-able part is introduced into the cavity where a biopsy sample has been removed from a tumor.

Using a biopsy procedure to make one or more cavities in a tumor would enable personalized inductive heated hyperthermia treatment of any cell growth or infection in the body without delay without stressing the rest of the patient's body.

FIG. 1C illustrates a method for conducting a biopsy procedure and insert delivery 100C. The method includes a step of inserting a needle in a tumor 122. Cutting a sample from the tumor and a delivery is inserted 124. The sample is collected in an aperture of the needle and removed from the patient 126.

The insert part could also be coated in a specific protein or other kind of bait e.g., amino acid and/or sugars targeted e.g., at cancer cells and applied directly into e.g., a stent into the blood vessel for leukemia and/or directly into a cancer tumor where the cancer cells that then would try to engulf them and be ready for cell interaction when exposed to induction heat.

Controlling size and material composition of the heat-able element along with distance to the inductive heat source and the level/strength of eddy current are important features.

The delivering device for the heat-able parts could be similar to a biopsy device with the extra feature being able to deliver the heat-able part at a desired destination.

Image-guided biopsy combines an imaging procedure such as a CT scan, MRI or ultrasound with a biopsy needle and can be used as part of the delivery device of the heat-able part.

This method and device could also be used as a medical treatment delivery vehicle system to deliver hyperthermia, targeted chemotherapy or localized radiation treatment.

The use of uniformly sized macro particles on as the heat-able elements (such as the signs size of a grain of rice), rather than nanoparticles allows the more accurate regulation of temperature and position of treatment.

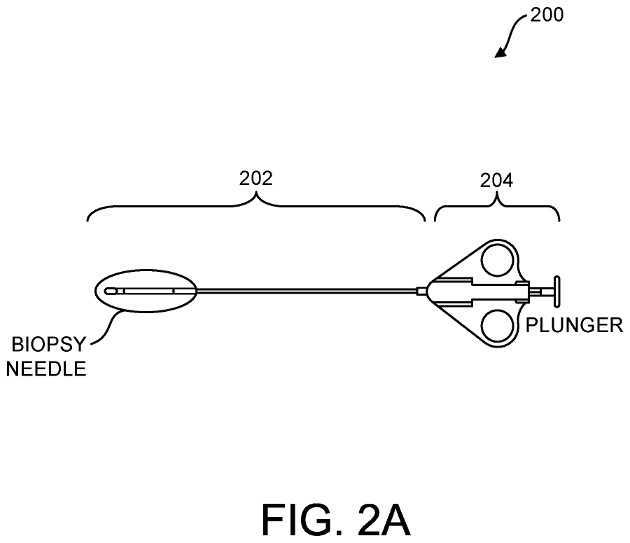

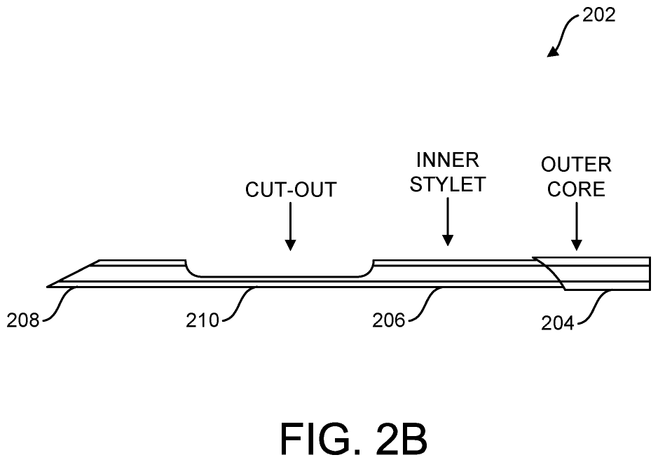

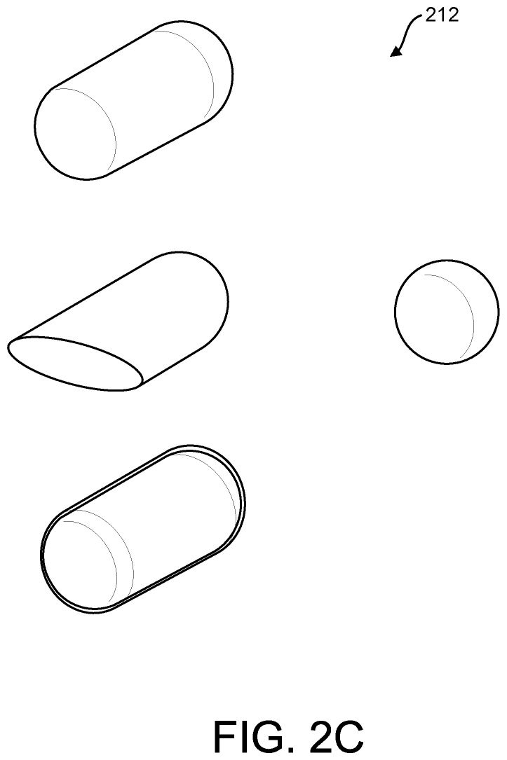

FIG. 2A illustrates a surgical tool 200 to conduct biopsy. The surgical tool includes a needle portion 202 and a plunger portion 204. The needle portion 202 includes two positions a closed position and an open position. The needle portion 202 is inserted and removed in closed position and the sample is removed in open position.

The plunger 204 is basically the handle of the surgical tool to control the movement of the surgical tool and to change the position of the needle portion.

An alternative to the biopsy needle could be an endoscope procedure. The endoscope can be inserted through your mouth, rectum, urinary tract or a small incision in your skin, and the endoscope having a flexible tube with a camera to see structures inside the body capable of introducing special tools through the tube to deliver the heat-able part at the desired destination.

Another alternative could be a surgical incision to access the suspicious area of cells to deliver the heat-able part at the desired destination.

FIG. 2B illustrates a side view of a needle portion 202 of the surgical tool. The needle portion 202 of the surgical tool includes an outer core 204 and an inner stylet 206. The outer core 204 is an outer layer i.e. retrieved a little to expose an inner layer when the position of the surgical tool is confirmed.

The device is used as a medical treatment delivery vehicle system to deliver parts for hyperthermia, targeted chemotherapy, or localized radiation treatment.

FIG. 2C illustrates a number of variations in delivery unit of the needle portion. The delivery unit 212 can be a capsule-shaped, needle-shaped, a spherical and can be coated with a specific protein or other kind of bait (e.g., amino acid and/or sugars) targeted at cancer cells.

The insert comprises a conductive core, such as a metal, copper, aluminum, steel, or brass. The material can also be a semiconductor such as graphite, carbon, silicon carbide, metal oxide or a compounded material.

Moreover, having self-controlling low Curie temperature magnetic heat able part material for induction heating based on compounding mix. The magnetization decreases with increasing amount of additives thereby controlling the saturation magnetization. The compositions of e.g. alloys can be mixed to realize a suitable Curie temperature.

Controlling size and material composition of the heat-able element along with distance to the inductive heat source and the level/strength of eddy current are important features.

Multiple inductive heat activations could be done to insure the drug release. The inductive heat activations could be activated by a sensor e.g. a blood sugar level sensor or sleep sensor with at least one activation of the inductive heating device ensuring that the activation of the drug delivery will be released in the intestine. This could also be done when no ultrasound or other detection technology is used to confirm the position the drug delivery part, by e.g. a preset repeated inductive heating activation from a induction device positioned below the hostile environment of the stomach. This would ensure that the inductive heating activation of the drug delivery part takes place outside the hostile environment of the stomach where the drug could be ruined by stomach acid. This could also compensate for different travel time through the body to reach the intestine that often is a problem in current time release drug delivery products. The oral delivery time release part could also have more than one drug compartment thereby enabling a mixing and release of at least two drug components as the inductive heating device is activated. This would be a benefit where drug component had a limited shelf time after being mixed. The steps of the drug release could then be that the wall between the at least two drug compartments opened for mixing before the final release into the intestine.

The drug delivery could also be targeted at lifestyle decease treatments like diabetes and obesity. Where the inductive heating device is activated on a to need basis dictated by blood sugar level and/or other indicator e.g. like a sleep sensor, but could also be on a predetermined schedule with at least one activation of the inductive heating device ensuring that the activation of the drug delivery will be released in the intestine when no ultrasound or other detection technology is used. By having multiple inductive heating activations in a targeted area of the body where the desired place of release the is, it could e.g. be insured that the drug delivery part had reached the desired intestine area before it was activated. The inductive heating device could e.g. be placed around the waist like a belt and/or belt buckle positioned below the hostile environment of the stomach thereby ensuring that the inductive heating activation of the drug delivery part takes place outside the hostile environment of the stomach where the drug could be ruined by stomach acid. Such a belt version could be used for home treatment to be worn on daily and/or nightly to insure continues treatment especially on lifestyle diseases.

While illustrative implementations of the application have been described in detail herein, it is to be understood that the inventive concepts may be otherwise variously embodied and employed, and that the appended claims are intended to be construed to include such variations, except as limited by the prior art.

Reference throughout this specification to “one implementation” or “an implementation” means that a particular feature, structure, or characteristic described in connection with the implementation is included in at least one implementation of the present invention. Thus, the appearances of the phrases “in one implementation” or “in some implementations” in various places throughout this specification are not necessarily all referring to the same implementation. Furthermore, the particular features, structures, or characteristics may be combined in any suitable manner in one or more implementations.

Systems and methods describing the present invention have been described. It will be understood that the descriptions of some embodiments of the present invention do not limit the various alternative, modified, and equivalent embodiments which may be include within the spirit and scope of the present invention as defined by the appended claims. Furthermore, in the detailed description above, numerous specific details are set forth to provide an understanding of various embodiments of the present invention. However, some embodiments of the present invention may be practiced without these specific details. In other instances, well known methods, procedures, and components have not been described in detail so as not to unnecessarily obscure aspects of the present embodiments.

1. A method of delivering at least one part capable of extended contactless treatment of targeted cells for an extend period after the placement of said part;
introducing the part, for extended contactless treatment of targeted cells into the body of a living being by means of a surgical tool, wherein the method comprising:
first performing at least one incision in the area of the targeted cells with a surgical tool, placing the surgical tool in a position within patient body for delivering of a part;
confirming the position of the surgical tool in the area of the targeted cells through an ultrasound or other detection technology;
releasing the part contained by the surgical tool in the cavity made by the surgical tool; and
removing the surgical tool from the patient body enabling a contactless extended targeted cell treatment after the delivery procedure.

2. The method as in claim 1, wherein the delivered part is made of a material capable of inductive contactless heating enabling a personalized contactless inductive heated hyperthermia treatment of a cell growth or infection in the patient body without delay and without stressing the patient body unnecessarily.

3. The method as in claim 1, wherein the delivered part is made of a material/compound capable of a slow dissolving time release drug delivery enabling a continues drug delivery over an extended period giving personalized targeted treatment of a cell growth or infection in the patient body without delay and without stressing the patient body unnecessarily.

4. The method as in claim 1, wherein the delivered part is made of a material/compound capable of releasing a low targeted radiation thereby enabling a continues treatment over an extended period giving personalized targeted treatment of a cell growth or infection in the patient body without delay and without stressing the patient body unnecessarily.

5. The method as in claim 1, wherein the surgical tool can perform a biopsy procedure collecting a sample while delivering the part for extended contactless treatment of targeted cells into the body of a living being.

6. The method as in claim 1, wherein the conductive part is coated with a bait targeted at cancer cells and applied as a stent directly into blood vain for leukemia or in another desired shape into the cancer tumor or close by the cancer tumor for hyperthermia treatment.

7. The method as in claim 6, wherein the cancer cells try to engulf them and be ready for cell interaction when exposed to induction heat.

8. The method as in claim 1, where the inserted part is biodegradable in full or in part.

9. The method as in claim 1, where the inserted part is made of compounded materials capable of at least one of the disclosed treatment options e.g. inductive heating and/or radiation and/or time release drug delivery in one or more combinations.

10. A surgical tool for conducting a biopsy, wherein the surgical tool comprising:
a needle portion, wherein the needle portion includes:
an outer core, wherein the outer core is an outer layer retrieved a little to expose an inner layer when the position of the surgical tool is confirmed;
an inner stylet, wherein the inner stylet has a compartment containing a macro sized induction heat-able part, further wherein the inner stylet includes:
an aperture, wherein the aperture is a cutout to collect a sample; and
a plunger portion, wherein the plunger portion is connected to the base of the needle portion to control the movement of the needle portion inside the patient during the biopsy procedure.

11. The system as in claim 10, wherein the device is used as a medical treatment delivery vehicle system to deliver hyperthermia, targeted drug delivery like e.g. chemotherapy, or localized radiation treatment.

12. A method of contactless treatment of a targeted medical condition for treatment of a targeted medical condition by:
introducing an oral delivery time release part into a patient body;
placing an inductive heating device at a position on said patient body for heat activation of the time release part; and
activating the inductive heating device to activate the time release part to deliver a drug contained in the time release part.

13. A method of contactless treatment as in claim 12, including the step of confirming the position of the time release part in a targeted delivery area through an ultrasound or other detection technology.
